SEQUENCE LISTING (1 sequence)

SEQ ID NO: 1 - protein, 23 aa, synthetic construct
  REGION 1..23: note = Synthetic Peptide
RPPGFTPFRKIFWLKLGKLIDAL

(19) United States
(12) Patent Application Publication
(10) Pub. No.: US 2023/0293420 A1
(43) Pub. Date: Sep. 21, 2023

(54) COSMETIC OR DERMOPHARMACEUTICAL COMPOSITION, PEPTIDE AND USE IN THE REDUCTION AND ELIMINATION OF WRINKLES AND EXPRESSION LINES

(71) Applicants: NANOCEUTICALS LTDA, Goiânia (BR); BIOINTECH BIOTECNOLOGIA LTDA, Brasília (BR)

(72) Inventors: JOÃO DAIVISON SILVA RAMALHO, Brasilia (BR); GUILHERME ALVES FERREIRA, Aparecida de Goiânia (BR); NICHOLLAS SERAFIM CAMARGO, Brasilia (BR)

(73) Assignees: NANOCEUTICALS LTDA, Goiânia, GO (BR); BIOINTECH BIOTECNOLOGIA LTDA, Brasília, DF (BR)

(21) Appl. No.: 18/296,032

(22) Filed: Apr. 5, 2023

(30) Foreign Application Priority Data
Feb. 8, 2022 (BR) 10 2022 002377 8

(51) Int. Cl. A61K 8/64; C07K 14/00; A61K 8/9789; A61K 8/49; A61Q 19/08

(52) U.S. Cl. CPC A61K 8/64; C07K 14/001; A61K 8/9789; A61K 8/49; A61Q 19/08

(57) ABSTRACT

The present invention relates to an anti-wrinkles and expression lines peptide (Giovian (SEQ ID N^{o}1)), a cosmetic or dermopharmaceutical composition containing this peptide as an active ingredient, and the use of this composition in the treatment of wrinkles and expression lines. The composition works through muscle relaxation and reduction of the contractile activity of dermal cells. Since it is a cosmetic composition, with non-invasive topical application, it can be used on a daily routine without the need of cosmetology specialist, in addition to not being costly as the techniques currently known for treating wrinkles and expression lines.

The present invention relates to an anti-wrinkle and expression line attenuator peptide (Giovian (SEQ ID No. 1)), a cosmetic or dermopharmaceutical composition containing this peptide as an active ingredient, and the use of this composition in the treatment of wrinkles and expression lines. The composition acts through muscle relaxation and in reducing the contractile activity of dermal cells. Since it is a cosmetic composition, with non-invasive topical application, it can be used on a daily routine without the need for a specialist in the field of cosmetics, in addition to not being costly as the techniques currently known for treating wrinkles and expression lines.

With advancing age, elasticity is lost due to a reduction in the constitution of the extracellular skin matrix, represented by a reduction in collagen and elastic fibers in the skin. Such changes can be accompanied by an increase in the contractile activity of skin cells, which together promote some aesthetic changes compatible with skin aging, markedly represented by the appearance of wrinkles and expression lines.

Despite being related to aging, the appearance of wrinkles can be intensified by some unhealthy habits, such as smoking, poor diet, excessive exposure to the sun without the use of sunscreen, reduced water intake, insufficient sleep periods, among others. Heredity is also an aspect that can influence the tendency to develop wrinkles.

Anti-wrinkle treatment can be started from the age of 25, with creams and daily care, while aesthetic treatments can be started from the age of 30-35, when skin sagging becomes evident.

The options for aesthetic treatments have increased more and more, with the emergence of different anti-wrinkle and attenuation of expression lines treatments. For the case of finer expression lines and wrinkles, anti-wrinkle or anti-aging creams stand out, in addition to manual therapy techniques that mobilize facial muscles, radiofrequency, microneedling, and the use of botulinum toxin (Botox), whose function is to block the contractile activity of dermal cells.

For the treatment of deeper wrinkles, which remain even when the skin is stretched, the most applied treatments are acid peeling, HeNe laser, filling with hyaluronic acid, platelet-rich plasma, and even plastic surgery, such as a facelift.

However, many of these aesthetic treatments are not accessible to a large part of the population since they are expensive and due to the difficulty of adherence due to the high daily demand. Moreover, some of them are restricted to some groups of people.

Radiofrequency, for example, cannot be applied to people with signs of infection or inflammation in the area to be treated; it is not recommended for pregnant women, people who have hypertension or changes related to increased collagen production, such as keloids, for example (VIEIRA, Giovanna De Simone K. Importância da radiofrequência em tratamentos estéticos: revisão da literatura. Specialization Work, 2016. Pontifícia Universidade Católica de Goiás—Departamento de Fisioterapia) In addition, it is a technique that can put the patient at risk, as misuse of the equipment can cause skin burns.

Diamond peeling is not recommended for those with very sensitive, inflamed skin or with acne grades II, III or IV. In these cases, it is necessary to wait until the skin is healed and the dermatologist authorizes the procedure to avoid injuries (available at https://www.peleclinicadermatologia.com.br/blog/estetica/peeling-de-diamantes/access on 25 Jan. 2022).

Chemical peeling gives more satisfactory results only on lighter skin. In addition, post-peel care must be very strict (FIGUEIREDO, Vania et al. Chemical peels: review and practical applications. Surg Cosmet Dermatol. Vol. 5. 1.ed; 58-68, 2013; TRUCHUELO, M.; CERDÁ, P.; FERNANDEZ L. F. Peeling químico, una herramienta útil en la consulta. Academia Española de Dermatología y Venereología. Vol. 108. 4.ed; 315-322, 2016).

Currently, the use of botulinum toxin—Botox—is recurrent in the treatment of wrinkles, both expression and age wrinkles. Botox works by blocking the production or release of acetylcholine at synapses and neuromuscular junctions. For skeletal muscle to contract, acetylcholine is released from the nerves. Acetylcholine binds to its receptors in the muscle, causing its cells to contract. Therefore, Botox prevents the release of acetylcholine by the nerves, causing the muscle to no longer contract, that is, leaving it relaxed.

However, the use of Botox can cause some adverse effects and complications arising from the injection or the product itself.

Among the complications, there is palpebral ptosis, which can be caused by very high dilutions of the toxin, injections very close to the orbital rim, massages or intense manipulation of the area after application and by the greater diffusion of toxin preparation. Difficulty closing the eyelids (lagophthalmos) may also occur in the treatment of periorbital wrinkles; superciliary ptosis and a significant decrease in the expressiveness of the upper third of the face; another unwanted effect is lateral eyebrow ptosis (ZAGUI, R. M. B.; MATAYOSHI, S.; MOURA, F. C. Efeitos adversos associados à aplicação de toxina botulínica na face: revisão sistemática corn meta-análise. Arq Bras Oftalmol. v. 71, n. 6, p. 894-901, 2008). Asymmetrical spots may also appear on the face; such as ptosis of the upper lip, in addition to the difficulty in moving the lower lip, causing damage to the bite and speech (MAIO, Maurício. Tratato de Medicina Estética. 2.ed, v. 2, Sao Paulo: Roca, 2011.).

It should also be taken into account that the effectiveness of Botox is only 3 to 6 months, after this period, the injections must be repeated so that the effect is maintained, running the risk of increasing adverse effects.

Additionally, any aesthetic treatment comes with a risk, so the professional working in dermatology must have full knowledge of the anatomy, muscles and subcutaneous tissue of the face. (SANTOS, Thiago José. Aplicação da toxina Botulínica ern Dermatologia e estética e suas complicações: Revisão da Literatura. Trabalho de obtenção de título de pós-graduação ern Dermatologia—Núcleo Alfenas, 2013).

Therefore, the present invention consists of an option for the treatment of wrinkles through a simple cosmetic or dermopharmaceutical composition, for non-invasive topical application, preferably on the skin of the face, and which can be used on a daily basis without the presence of a professional, being efficient both in the treatment of finer wrinkles and those deeper. The composition of the present invention also works by reducing the contractility of dermal cells in a non-invasive manner. Advantageously, the active principle, the peptide (called GIOVIAN), can be absorbed by the skin, so the composition can be presented in the form of a cream, serum, or other pharmaceutical form for topical application on the skin, being easily used in everyday life, and saving the individual from taking risks with aesthetic techniques. In addition, it is a simple, less expensive dermofacial treatment that may provide greater adherence by the individual when compared to the aforementioned methods.

In searches carried out in patent banks, documents were found that describe anti-wrinkle compositions comprising peptides, such as document WO2017183942A2 that describes a composition containing peptide that is effective in increasing collagen production, thus reducing wrinkles and preventing age marks. Document KR20170050327A describes an anti-aging cosmetic composition comprising keratin peptide as an active compound. Document EP3904369A1 describes a new peptide that has the activity of preventing damage caused to the skin by pollutants, alleviating wrinkles and with anti-aging activity. The peptide prevents the influx of pollutants into the skin and can prevent and treat cancer, skin and lung diseases. Document KR20200101765A refers to a peptide with anti-aging skin activity and a composition containing the peptide, which exhibits physiological activity such as inhibition of collagen degradation, and therefore may be useful for wrinkle reduction, skin regeneration, improving skin elasticity, inhibiting skin aging, wound regeneration and the like. WO2013133482A1 refers to a method for extracting mandarin wasp venom to produce a functional cosmetic composition. Document CN101352402A describes a rejuvenating dual nutrition massage cream containing bee peptide, as well as the method for producing it. Document US2009111731 describes new agents for topical application against mimic and age-related wrinkles.

However, the composition as well as the peptide presented in the present invention presents a sequence of amino acids different from those found in the state of the art, in order to configure an unprecedented composition. The composition of the present invention acts by inhibiting the neuromuscular synapse, muscle contraction, thus resulting in the reduction of wrinkles and the attenuation of expression lines.

### BRIEF DESCRIPTION OF THE FIGURES

FIG. 1—Percentage of cell viability for treatment with the Giovian sample (SEQ ID No. 1).

FIG. 2—Representative images of each group after treatment. The negative control, without the presence of cells, did not show a reduction in volume, that is, there is no cell contractility. The images represent a collagen disc containing a population of dermal cells with contractile activity. The experiment consists of the evaluation of the contractile activity, by measuring the change in the volume of the collagen disc.

FIG. 3—Effect of the peptide concentration curve on cell relaxation.

FIG. 4—Evaluation of Cosmetic Appreciability in Clinical Tests.

FIG. 5—Evaluation of Subjective Clinical Efficacy after 30+/−2 days.

FIG. 6—Perception after using the composition.

### DETAILED DESCRIPTION OF THE INVENTION

The present invention is a cosmetic or dermopharmaceutical composition containing the peptide (Giovian—SEQ ID No. 1) and its use in reducing and eliminating wrinkles and expression lines.

The peptide of the present invention (SEQ ID No. 1) has a theoretical mass of 2713.20 (purity >95%) and it has the following peptide sequence: H-Arg-Pro-Pro-Gly-Phe-Thr-Pro-Phe-Arg-Lys-Ile-Phe-Trp-Leu-Lys-Leu-Gly-Lys-Leu-Ile-Asp-Ala-Leu-NH2.

Also, for the present invention, sequences in which the amino acid Isoleucine is replaced by the amino acid Leucine with the same chemical property and vice versa can be considered.

The formulation containing the peptide consists of water, hyaluronic acid 0.2% to 1%, Giovian peptide (SEQ ID No. 1) 0.0002% to 0.005%, and/or peptides obtained by replacing the amino acids Isoleucine by Leucine in the structure of SEQ ID No. 1; black wattle tannin 0.01% to 0.25%, Isothiazolinone 0.02% to 0.5%.

In vitro cytotoxicity studies were performed, considering eukaryotic dermatological cell culture systems; as well as the ability of myofibroblasts to contract in cell culture systems on collagen discs to verify the Botox-like activity of the Giovian (SEQ ID No. 1). Regarding clinical trials, trials were carried out with volunteer individuals (n=36, CEP/Plataforma Brasil number 5,121,752) in order to evaluate the safety and efficacy profile of Giovian (SEQ ID No. 1). In addition, a Giovian (SEQ ID No. 1) perceived functionality analysis was also analyzed in the clinical trials performed.

The technology can be better understood from the following non-limiting examples.

### Example 1—In Vitro Biological Assays Using Eukaryotic Dermal Cell Systems in Cell Culture

1.1. In Vitro Study of Neutral Red Cytotoxicity

### Objective:

Evaluate the in vitro cytotoxic potential of the Giovian peptide (SEQ ID No. 1) in culture of Balb/c 3T3 clone 31 cells.

### Methodology:

### Cell Culture:

In this study, Balb.c 3T3 clone 31 cells were used, obtained from the supplier Banco de Células do Rio de Janeiro, batch 001242, maintained in cultivation with DMEM (Dulbecco's Modified Eagle's Medium) with the addition of supplements, in an oven at 37° C. and 5% of CO2, and were manipulated inside the laminar flow hood. In passage 04 after thawing, the cells were distributed in 96-well plates maintained in culture under the same conditions described.

### Preparation of Giovian (SEQ ID No. 1) Sample:

Sample preparation conditions in the starting solution: sample was diluted to 1 mg/mL in medium and cell culture;
Preparation of concentrations: the first concentration tested was 1 mg/mL and the following concentrations were diluted with 10% from the previous concentration in supplemented cell culture medium;
After the first evaluation, a second study was conducted with the following concentrations: 0.1, 0.09, 0.075, 0.06, 0.05, 0.0375, 0.025 and 0.01 mg/mL; Appearance of solubilized sample: fully solubilized.
Preparation of control groups: Control group: supplemented cell culture medium; Positive control group: sodium lauryl sulfate diluted in supplemented cell culture medium.
Incubation and assessment of cell viability. The solutions containing the control, positive control and sample groups were applied to the cell culture, 100 μL per well in quadruplicate for each group, followed by incubation in an oven at 37° C. and 5% CO₂ for 24 hours. After washing, cell viability was analyzed with the neutral red indicator, 100 μL per well. After incubation, the reaction was revealed, and absorbance was determined at 540 nm.

### Results:

The results were evaluated using Microsoft Excel software. The control group was normalized to 100% and the percentage of cell viability in the sample and in the positive control was calculated in relation to the control. Samples are considered cytotoxic when they have 30% or more reduction in cell viability.

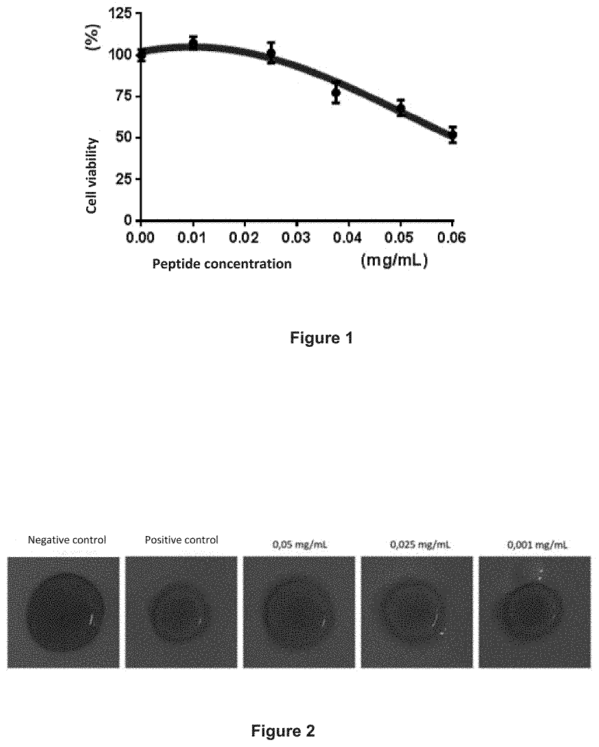

According to the results obtained (FIG. 1), it is possible to affirm that the sample of Giovian (SEQ ID No. 1) did not present cytotoxic effect at concentrations lower than 0.0375 mg/ml.

1.2. In Vitro Study of Botox-Like Effect—Cell Contractility In Vitro

### Objective:

Evaluate the potential of the sample to reduce cell contractility using myoblast cells.

### Methodology:

### Cell Culture:

In this study, myoblast cell cultures were used, maintained in cultivation with DMEM (Dulbecco's Modified Eagle's Medium) with the addition of supplements, in an oven at 37° C. and 5% CO2 and manipulated inside a laminar flow hood. To stimulate contraction of these cells, they were embedded in a three-dimensional collagen matrix. After the polymerization of the matrix soaked with the myoblasts, the culture medium was added according to the experimental groups. For the negative control group, a three-dimensional matrix was made without the presence of cells. All groups were performed in triplicate.

### Preparation of Giovian (SEQ ID No. 1) Sample:

Sample preparation conditions in the starting solution: 1 mg/ml in culture medium; Evaluated concentrations: 0.05; 0.025 and 0.01 mg/ml;
Control group: culture medium; Negative control group: absence of cells in the collagen gel;
Incubation and evaluation of contractility. After adding the culture medium in the control group and the culture medium containing the Giovian sample (SEQ ID No. 1), the gels were kept in an oven for 72 hours. At the end of time, each matrix was measured to calculate the final volume, and photographs were taken of each evaluated matrix.

### Results:

The results were evaluated using Graphpad Prism 5 software. The groups were compared and statistically analyzed by the test OneWay Anova followed by the Bonferroni test and established as a statistical difference when p was less than 0.05.

FIG. 2 shows the macroscopic appearance of collagen discs containing myofibroblasts treated with different concentrations of Giovian (SEQ ID No. 1). It is possible to observe the differential circumferential dimension pattern in each experimental group. The negative control that does not present cells did not demonstrate volume reduction, that is, there is no cell contractility.

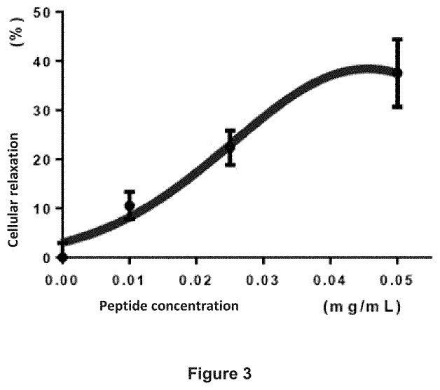

The higher the doses of Giovian (SEQ ID No. 1), the greater the allos, which demonstrates the increase in cell relaxation, that is, the cells reduce contraction, thus promoting relaxation from the effect of the peptide. From the dimensional analysis of the results shown in FIG. 2, qualitative evidence of the contractility capacity of the Giovian (SEQ ID No. 1) peptide can be constructed. The results of FIG. 2 are quantitatively represented in FIG. 3, where it is possible to observe a graphical dose-response profile of the peptide activity in cell relaxation. With this evidence, it is possible to prove the activity of inhibiting the contractility of dermal cells by Giovian (SEQ ID No. 1), which would have the consequence of reducing the “wrinkling” aspect of the skin in clinical situations. That is, the greater the cell relaxation, the better the effect of reducing wrinkles and expression lines clinically detected.

### Example 2—Subjective Evaluation of Clinical Anti-Wrinkle Effectiveness, with Cosmetic Appreciability and Dermatological Acceptability

With the aim of evaluating the safety and efficacy of Giovian (SEQ ID No. 1) in humans, clinical trials were carried out with 32 volunteers, aged 35 to 65 years, who used the product containing the peptide for 30 days.

No participant reported discomfort sensations and no clinical signs were detected after using the product. Therefore, it supports the “dermatologically tested” appeal.

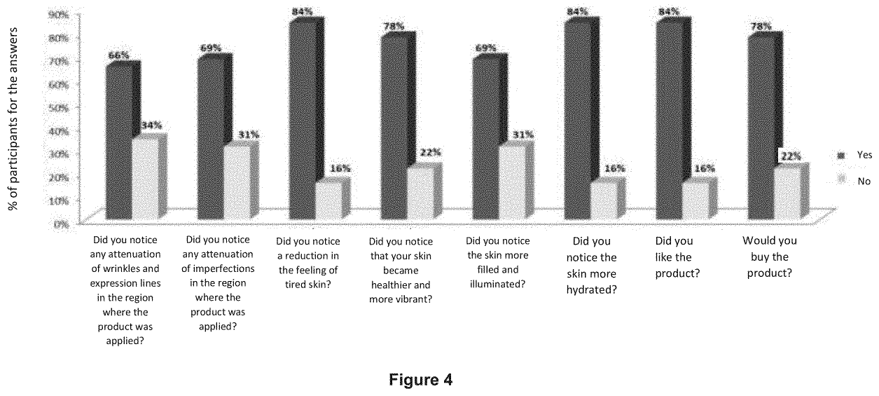

A cosmetic appreciability questionnaire was applied after 30+/−2 days of product use. In the questionnaire, some perceptions of the volunteers were evaluated, based on answers with “Yes” or “No”. Below, the evaluated questions and the percentage of positive and negative responses to the questions are listed (FIG. 4).

If there was attenuation of wrinkles and expression lines in the region where the product was applied;
Was there attenuation of imperfections in the region where the product was applied?
66% Yes/34% No
Was there a reduction in response to the feeling of tired skin?
69% Yes/31% No
Has the skin become healthier and fresher?
84% Yes/16% No
Has the skin become fuller and brighter?
78% Yes/22% No
Has the skin become more hydrated?
69% Yes/31% No
Did the volunteer like the product?
84% Yes/16% No
Would the volunteer buy the product?
78% Yes/2% No

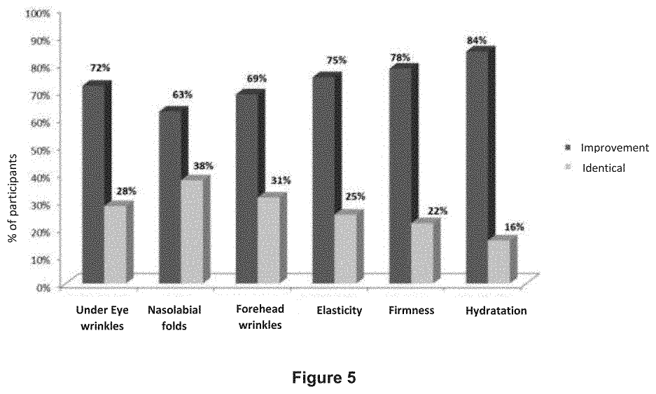

The subjective clinical efficacy of the composition was also evaluated after 30+/−2 days of use (FIG. 5). It was evaluated whether there was an improvement or if it remained the same: crow's feet wrinkles, nasolabial folds, forehead wrinkles, skin elasticity, skin firmness, and hydration. 72% of participants saw improvement in crow's feet wrinkles, 63% saw improvement in nasolabial folds, 69% saw improvement in forehead wrinkles, 75% saw improvement in elasticity, and 78% saw improvement in skin firmness.

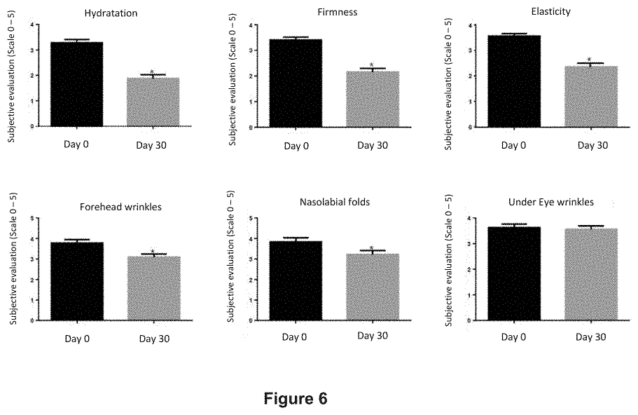

FIG. 6 shows the response of the participants, considering a scale from 0 to 5, regarding the effects of the composition on improving Hydration, Firmness, Elasticity, forehead wrinkles, nasolabial folds, and crow's feet wrinkles.

From the results presented, it can be seen that the composition of the present invention has clinical efficacy in improving wrinkles, lip juices, skin elasticity and firmness.

1. A peptide wherein it presents the sequence SEQ ID No: 1.

2. The peptide according to claim 1, wherein it presents the sequence SEQ ID N^{o}1 with the replacement of the amino acid Ile by the amino acid Leu and vice versa.

3. The peptide according to claim 1,
wherein it can be used in the preparation of anti-wrinkle cosmetic or dermopharmaceutical compositions and expression lines attenuation.

4. Cosmetic or dermopharmaceutical composition wherein it consists of water, hyaluronic acid from 0.2% to 1%, peptide of SEQ ID No. 1 from 0.0002% to 0.005%; black acacia tannin from 0.01% to 0.25%, and Isothiazolinone from 0.02% to 0.5%.

5. Cosmetic or dermopharmaceutical composition according to claim 4, wherein the peptide of the composition may result from replacements of the amino acids Ile, SEQ ID No. 1, by the amino acid Leu and vice versa.

6. Use of the cosmetic or dermopharmaceutical composition according to claim 4, wherein it is used on the skin for the reduction and elimination of wrinkles and expression lines.

7. The peptide according to claim 2, wherein it can be used in the preparation of anti-wrinkle cosmetic or dermopharmaceutical compositions and expression lines attenuation

8. Use of the cosmetic or dermopharmaceutical composition according to claim 5, wherein it is used on the skin for the reduction and elimination of wrinkles and expression lines.
